(19) United States
(12) Patent Application Publication
(10) Pub. No.: US 2009/0087384 A1
(43) Pub. Date: Apr. 2, 2009

(54) MEDICINE AND METHOD FOR TREATMENT OF PROSTATE CANCER

(76) Inventors: Sueleyman Erguen, Essen (DE); Jens Fehre, Hausen (DE); Ralf Nanke, Neunkirchen am Brand (DE); Bernhard Singer, Essen (DE); Martin Stetter, Muenchen (DE); Derya Tilki, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,277

(22) Filed: Sep. 3, 2008

(30) Foreign Application Priority Data
Sep. 3, 2007 (DE) 10 2007 041 832.0

(51) Int. Cl. A61K 49/22; A61K 31/337

(52) U.S. Cl. 424/9.52; 514/449

(57) ABSTRACT

The invention concerns a medicine for treatment of prostate cancer and a corresponding treatment method. The medicine can be supplied to a prostate tumor through the bloodstream, contains an active component and a binding component as well as ultrasound microbubbles. The binding component includes coupling molecules that specifically bind to a determined target molecule formed in the endothelium of tumor-associated blood vessels and that is typical of the tumor or the tumor stage. The active component contains at least one chemotherapeutic substance. The coupling molecules are bound to the outside of the microbubbles and active substance molecules are likewise bound to the outside of the microbubbles or are enclosed by the microbubbles.

### BACKGROUND OF THE INVENTION

1. Field of the Invention

The present invention concerns medicine for treatment of prostate cancer as well as a corresponding treatment method.

2. Description of the Prior Art

Due to a lack of suitable diagnosis methods for preliminary stages of prostate tumors, medicines that can be supplied to a prostate tumor via the circulatory system are administered only when the tumor has already progressed, for example is already in the angiogenesis stage. The recovery chances are then often correspondingly low.

### SUMMARY OF THE INVENTION

An object of the present invention is to provide a medicine and a treatment method with which the recovery chances are improved in the treatment of prostate cancer.

This object is achieved by a medicine according to the invention that contains an active component and a binding component as well as ultrasonic microbubbles. As used herein, “ultrasonic microbubbles” means bubbles that can be destroyed (burst) by the application of an ultrasound field. The binding component comprises coupling molecules that specifically bind to CEACAM-1. The active component contains at least one chemotherapeutic substance. The microbubbles act as carriers for target molecules and active substance molecules, so the former are bound to the outside of the ultrasonic microbubbles. This binding possibility also exists for the active substance molecules, but these can also be present within the ultrasonic microbubbles (thus surrounded thereby), so they are then protected from any negative effect during the transport to the target location. For example, such a medicine distributes in the body after intravenous administration. The coupling molecules present on the surface of the microbubble produce a targeted binding and accumulation in a tissue in which CEACAM-1 is present. The invention is based on research results from Tilki et al. (Oncogene (2006 25, 4965-4974) according to which CEACAM-1 occurs only in the epithelium in a healthy prostate but not in the blood vessels of the prostate. In an early phase of the tumor development—for example in the stage of hgPIN (high grade epithelial prostatic neoplasia)—in which the tumor has not yet formed its own vessels, the expression of CEACAM-1 in the prostate epithelium is regulated down while it is regulated up in the endothelium of adjoining small blood vessels of the healthy tissue. This effect is utilized according to the invention to detect an early cancer stage. In contrast to a healthy prostate, a completely different accumulation (uptake, enrichment) image results given the presence of a tumor in the early stage. A concentration of the contrast agent in the region of the tumor occurs in the tumor tissue so that the tumor can be observed or diagnosed using a theranostic technique and can be therapeutically treated in a targeted manner by externally induced active substance release.

The active substance release within the tumor tissue is enabled by microbubbles (thus extremely small bubbles formed from a lipid double membrane, for example) that can be destroyed by ultrasound being used as carriers for the active component. The point in time of the active substance release can be freely selected by the treating physician by charging the tumor with ultrasound, advantageously via a rectal probe. Due to the accumulation, a highly therapeutically effective active substance concentration is achieved in the prostate tumor without cells of healthy tissue being affected by this to a noteworthy degree. In all cases, only a portion of the active substance molecules are carried along by the blood stream given the release controlled by ultrasound, so the concentration thereof is sharply reduced due to the distribution in the circulatory system. Furthermore, it is advantageous that the medicine simultaneously acts as a diagnostic agent since it allows detection of the prostate tumor. With use of the corresponding binding component according to the invention, early cancer stages can be detected and effectively treated in a targeted manner. The medicine thus can be used in theranosis, meaning that it serves both diagnostic and therapeutic purposes. Since the microbubbles also act as a contrast agent for ultrasound-based imaging methods, the accumulation of the medicine in the prostate can be tracked and the point in time of the active substance release by changing with ultrasound can be optimally selected. Moreover, in a treatment it is possible to monitor the success of preceding treatments with the use of an ultrasound-based imaging method.

The above statements analogously apply to a method according to the invention for treatment of prostate cancer in which an active substance release induced by ultrasound ensues after administration of the medicine and its accumulation in the prostate tumor.

### BRIEF DESCRIPTION OF THE DRAWINGS

FIG. 1 schematically shows a region of the prostate exhibiting a blood vessel and a tumor, the region exhibiting the accumulate of a medicine in the tumor according to the invention.

FIG. 2 is a representation corresponding to FIG. 1 that shows the principle of the targeted active substance release within the prostate according to the invention.

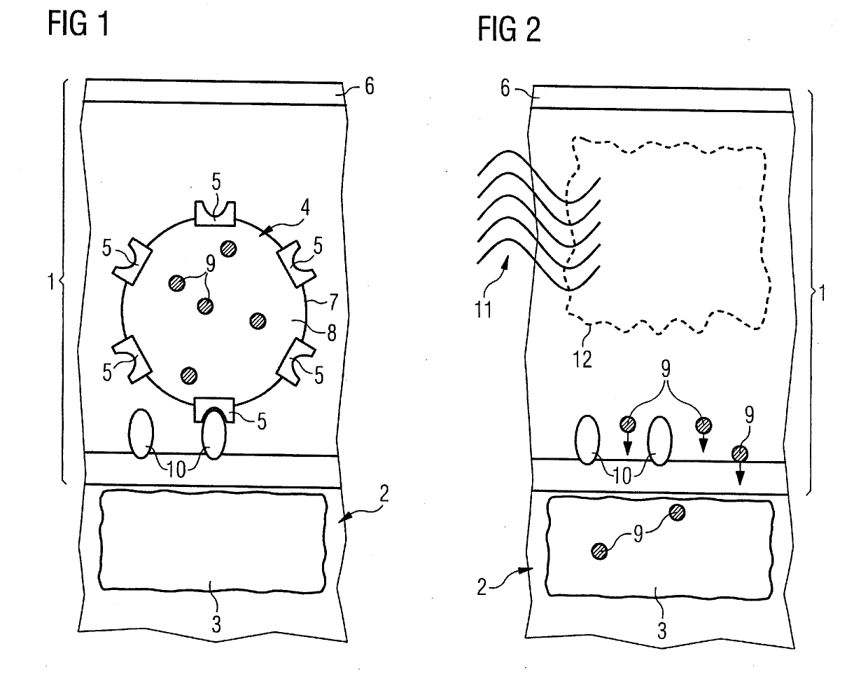

### DESCRIPTION OF THE PREFERRED EMBODIMENTS

A medicine supplied via a blood vessel 1 of the prostate 2 (and in particular of a tumor 3 present therein) contains a binding component, an active substance component and microbubbles 4 acting as carriers for the cited components. The binding component causes a targeted accumulate of the medicine. The binding component contains coupling molecules 5 that bind to CEACAM-1 (carcinoembryonic antigen-related cell adhesion molecule) as a target molecule 10. As explained above, these molecules are present in the endothelium of the blood vessels adjacent to the tumor located in an early stage. The coupling molecules are (indirectly or directly) bound to the outside 7 of the microbubbles. Microbubbles 4 are known. They form an envelope (formed from a lipid double membrane, for example) which surrounds a void 8.

The active components are active substance molecules 9 that, like the coupling molecules 5, are either bound to the outside of the microbubbles or enclosed in their voids 8. The latter variant guarantees that any interaction with components of the blood is precluded during the transport of the microbubbles in the blood stream. Moreover, a larger surface for accommodation of coupling molecules is available on the outside 7 of the microbubbles 4. In practice all therapeutic agents (such as docetaxel as well as active substances such as angiogenesis inhibitors, for example) suitable to combat the prostate tumor which slow or prevent the new formation of blood vessels can be considered as active substances. Due to the encapsulation of the active substances in the microbubbles, toxins can also be used that would damage the body given conventional application. The microbubbles can be loaded with only a single active substance or with multiple different, therapeutically active substances, or active substance molecules. As already mentioned, the microbubbles according to the invention fulfill a double function of serving as carriers for the active substance components as well as serving as a contrast agent for the imaging. The contrast enhancement can be attenuated given microbubbles 4 loaded (in filled) with active substance molecules, such that it is appropriate when the medicine contains microbubbles 4 that are free of active substance that do not differ from the microbubbles 4 carrying active substance molecules with regard to their binding action (i.e. the coupling molecules 5 present on their outsides).

The targeted accumulation in early stages of the prostate tumor 3 is produced by the respective binding partner (i.e. coupling molecules 5) by tissue-typical target molecules 10 (thus primarily CEACAM-1). In the early stage of the prostate tumor, for example the stage of high grade epithelial prostatic neoplasia (hgPIN), this has not yet developed any blood vessels 1. However, as a result of the tumor adjacent blood vessels form CEACAM-1 molecules in the endothelium. A medicine that contains coupling molecules specifically binding to CEACAM-1 (in particular CEACAM-1 antibodies) can thus detect hgPIN, for example, and accumulate in the corresponding tissue in a targeted manner so that this stage is made visible with ultrasound imaging. Further-advanced carcinomas induce angiogenesis, the formation of blood vessels 1. The growth factors accompanying angiogenesis (such as VEFG—vasco endothelial growth factors—for example) thereby serve as target molecules 10 with which corresponding coupling molecules 5 interact as binding partners (for example VEGF ligands or antibodies). In the cases previously cited, aptamers, spiegelmers or anticalins can also serve as coupling molecules. Aptamers are short, stable and specifically binding RNA chains, spiegelmers are the mirror-image counterparts of the aptamers. The anticalins are individual polypeptide chains with approximately 180 amino acids that possess specific binding properties similar to antibodies but can be produced more easily than these.

Even in a later cancer stage it can be appropriate to use a medicine that contains a portion or a charge of microbubbles whose outside 7 is provided with coupling molecules 5 that bind to CEACAM-1. For example, such a CEACAM-1-imparted marking helps to determine the proportion of angiogenetically active blood vessels in the tumor vascular bed or, respectively, to be able to detect the reaction of the tumor vessels to an anti-angiogenetic therapy in the imaging. It is also possible for the administration of such medicine to be combined with a medicine that contains microbubbles 4 with coupling molecules binding to VEGF in order to be able to allow a certain differentiation of the vessels within the tumor vascular bed to be made with regard to their activity. A medicine can also be administered that contains both of the cited types of microbubbles, such that complex cancer stages are detected.

An ultrasound theranostic session can proceed as follows: A charge of molecularly marked microbubbles (thus microbubbles possessing specifically binding coupling molecules 5) laden with active substance are injected into the bloodstream of the patient. If the patient has prostate cancer in an early or middle stage, the microbubbles selectively accumulate in the tumor 3 or in its surroundings in that their coupling molecules bind to endothelial CEACAM-1 molecules. Simultaneously, an ultrasound exposure of the prostate is made using an ultrasound apparatus or a rectal probe. The microbubbles thereby act in a contrast-enhancing manner. The accumulation process of the microbubbles thus can be tracked. Since the accumulation ensues only in the tumor, its presence and extent can be diagnosed. If additional theranosis sessions have already preceded the present session, this step simultaneously represents a course monitoring: by comparison with the preceding tumor images, so it can be established whether and to what extent the tumor has degenerated. If a tumor 3 is present and the accumulation process has proceeded sufficiently far, the physician causes an ultrasound pulse 11 to be emitted. This is acts to destroy the microbubbles (indicated by the dashed line 12 in FIG. 2). The active substance molecules 9 are thereby released into the tumor tissue in a precisely targeted and non-invasive manner and can deploy their effect there with maximum impact. Unbound microbubbles are located at other points in the body and are therefore not destroyed by the ultrasound pulse 11, such that healthy tissue remains protected from the effect of the active substance molecules 5.

Although further modifications and changes may be suggested by those skilled in the art, it is the intention of the inventors to embody within the patent warranted hereon all changes and modifications as reasonably and properly come within the scope of their contribution to the art.

We claim as our invention:

1. A medicine for treatment of prostate cancer, comprising:
an active component and a binding component and ultrasound microbubbles forming a combination that is administrable to a prostate tumor in a patient through the blood stream of the patient;
said binding agent being formed from coupling molecules that specifically bind to CEACAM-1;
said active component containing at least one chemotherapeutic substance; and
said coupling molecules being bound to an exterior of the microbubbles, and said microbubbles carrying said active substance molecules by said active substance molecules being bound to the exterior of the microbubbles or enclosed by the microbubbles, said microbubbles being destroyable upon application of ultrasound thereto.

2. A medicine as claimed in claim 1 comprising microbubbles that do not carry said active substance molecules.

3. A medicine as claimed in claim 1 wherein said active substance molecules are first active substance molecules, and wherein said medicine comprises second active substance molecules, different from said first active substance molecules, carried by ultrasound microbubbles.

4. A medicine as claimed in claim 3 wherein said second active substance molecules are selected from the group consisting of anti-angiogenic substances and chemotherapeutic substances.

5. A medicine as claimed in claim 1 wherein said coupling molecules are CEACAM-1 antibodies.

6. A medicine as claimed in claim 5 wherein said coupling molecules are selected from the group consisting of aptamers, spiegelmers and anticalins.

7. A medicine as claimed in claim 1 wherein said coupling molecules comprise coupling molecules that are typical of a later stage tumor following the hgPIN stage.

8. A medicine as claimed in claim 7 wherein said coupling molecules bind to the blood vessel growth factor VEGF.

9. A medicine as claimed in claim 8 wherein said coupling molecules are VEGF ligands or VEGF antibodies.

10. A medicine as claimed in claim 8 wherein said coupling molecules are selected from the group consisting of aptamers, spiegelmers and anticalins.

11. A method for treating prostate cancer comprising the steps of:
via the bloodstream of a patient having a prostate tumor, administering a medicine containing an active component and a binding component and ultrasound microbubbles, said binding component being formed from coupling molecules that specifically bind to CEACAM-1 and said active component including at least one chemotherapeutic substance and said coupling molecules being bound to the outside of the microbubbles and the active substance molecules being carried by the microbubbles at the outside thereof or enclosed by the microbubbles; and
destroying said microbubbles by applying an ultrasound pulse thereto.

12. A method as claimed in claim 11 comprising obtaining an ultrasound image of the prostate tumor after administration of said medicine thereto and before destruction of the microbubbles.
